(19) United States
(12) Patent Application Publication
(10) Pub. No.: US 2026/0114731 A1
(43) Pub. Date: Apr. 30, 2026

(54) PHYSIOLOGICAL CHARACTERISTIC MEASURING DEVICE

(71) Applicant: GUANGZHOU LUXVISIONS INNOVATION TECHNOLOGY LIMITED, Guangzhou City (CN)

(72) Inventor: Chih-Ju Lin, Guangzhou City (CN)

(73) Assignee: GUANGZHOU LUXVISIONS INNOVATION TECHNOLOGY LIMITED, Guangzhou City (CN)

(21) Appl. No.: 19/008,527

(22) Filed: Jan. 2, 2025

(30) Foreign Application Priority Data
Oct. 30, 2024 (CN) 202411526693.8

(51) Int. Cl. A61B 3/14; A61B 3/00

(52) U.S. Cl. CPC A61B 3/14; A61B 3/0025

(57) ABSTRACT

A physiological characteristic measuring device includes a light source module configured to provide incident linear polarized light with a first linear polarization toward an eye, a sensing module configured to sense light to be measured from the eye when the incident linear polarized light enters the eye, and a processing unit connected to the sensing module and providing a light intensity ratio, where the light intensity ratio is a ratio of a light intensity of a first linear polarized light of the light to be measured to a light intensity of a second linear polarized light of the light to be measured. The first and second linear polarized light has a second and first linear polarization respectively, and the second linear polarization is perpendicular to the first linear polarization. The processing unit provides a physiological characteristic measuring value according to an arc-tangent function of the light intensity ratio.

### CROSS-REFERENCE TO RELATED APPLICATION

This application claims the priority benefit of China application serial no. 202411526693.8, filed on Oct. 30, 2024. The entirety of the above-mentioned patent application is hereby incorporated by reference herein and made a part of this specification.

### BACKGROUND

### Technical Field

The invention relates to an optical measuring device, and particularly relates to a physiological characteristic measuring device.

### Description of Related Art

Diabetes is a global disease, but main blood glucose monitors (BGM) on the market currently require drawing blood, which increases discomfort of diabetic patients. In recent years, continuous glucose monitor (CGM) has been developed, which implants a glucose oxidase sensor with a soft needle under the skin to measure a subcutaneous blood glucose value to achieve a purpose of continuous detection.

Otherwise, most optical blood glucose measuring devices use analytical methods of Fourier transform infrared (FTIR) spectroscopy, Raman spectroscopy, and near infrared/middle infrared spectroscopy. However, the processing of drawing blood is still necessary.

Moreover, the above-mentioned optical measuring devices are difficult to be miniaturized and carried around.

### SUMMARY

The invention is directed to a physiological characteristic measuring device, which does not need to draw blood and is convenient to carry around.

An embodiment of the invention provides a physiological characteristic measuring device adapted to be arranged in front of an eye. The physiological characteristic measuring device includes a light source module, a sensing module and a processing unit. The light source module is configured to provide incident linear polarized light toward the eye, and the incident linear polarized light has a first linear polarization. When the incident linear polarized light enters the eye, the sensing module is configured to sense light to be measured from the eye. The processing unit is connected to the sensing module. The processing unit is configured to provide a light intensity ratio, where the light intensity ratio is a ratio of a light intensity of a first linear polarized light of the light to be measured to a light intensity of a second linear polarized light of the light to be measured, where the first linear polarized light has a second linear polarization, the second linear polarized light has the first linear polarization, and the second linear polarization is perpendicular to the first linear polarization. The processing unit provides a physiological characteristic measuring value according to an arc-tangent function of the light intensity ratio.

Another embodiment of the invention provides a physiological characteristic measuring device adapted to be arranged in front of an eye. The physiological characteristic measuring device includes a light source module, a sensing module and a processing unit. The light source module includes a light source and a first linear polarizer. The light source is configured to provide incident light toward the eye, and the incident light includes first incident light in a first time period and second incident light in a second time period. In the first time period, the first linear polarizer is not on a path of the incident light, and in the second time period, the first linear polarizer is on the path of the incident light. The sensing module includes a second linear polarizer. When the incident light enters the eye, the sensing module is configured to sense light to be measured from the eye to generate a light intensity of the light to be measured. The second linear polarizer is located on a path of the light to be measured, and an absorption axis of the first linear polarizer is perpendicular to an absorption axis of the second linear polarizer. The processing unit is connected to the sensing module. The processing unit is configured to provide a light intensity ratio, the light intensity ratio is a ratio of the light intensity of the light to be measured in the second time period to the light intensity of the light to be measured in the first time period. The processing unit provides a physiological characteristic measuring value according to an arc-sine function of the light intensity ratio.

Based on the above description, the physiological characteristic measuring device provided by the embodiment of the invention utilizes optical rotation of glucose and uses polarized light to measure a glucose concentration in aqueous humor of the eye to obtain a blood glucose concentration of a testee. Comparing the results with other invasive measuring devices, the physiological characteristic measuring device provided by the embodiment of the invention does not require drawing blood and is easy to carry around.

To make the aforementioned more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a structure of a human eye.

FIG. 2A is a schematic diagram of a physiological characteristic measuring device according to an embodiment of the invention.

FIG. 2B is a schematic diagram of a physiological characteristic measuring method according to an embodiment of the invention.

FIG. 3 is a schematic diagram of a physiological characteristic measuring device according to an embodiment of the invention.

### DESCRIPTION OF THE EMBODIMENTS

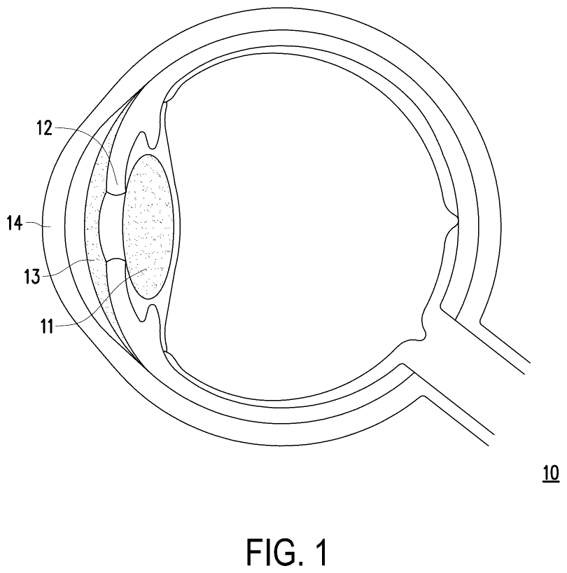

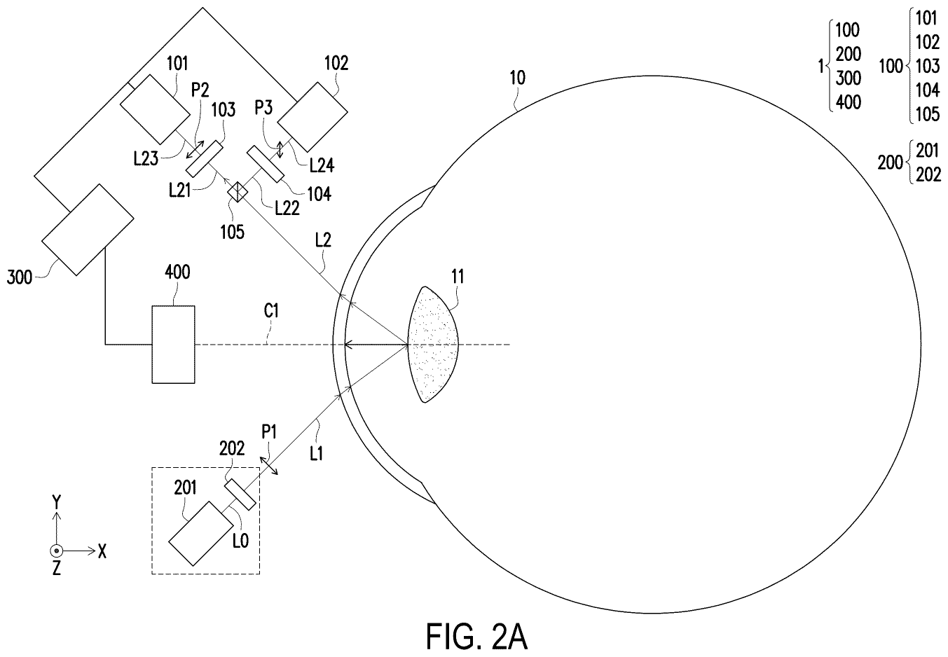

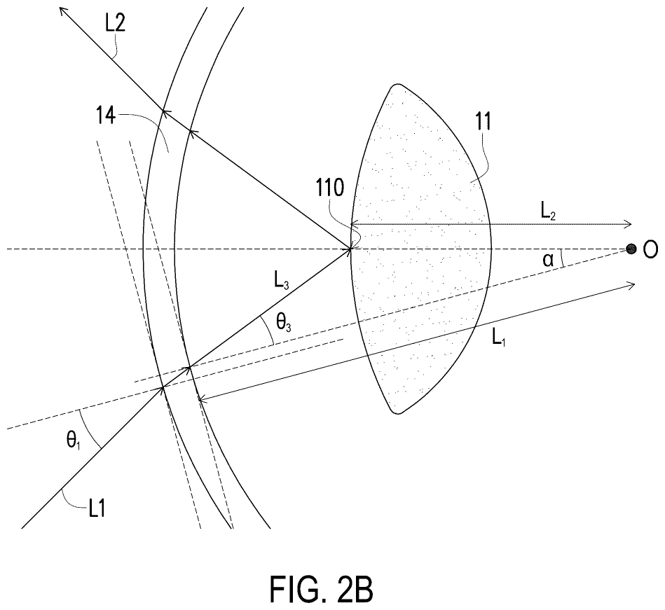

Referring to FIG. 1, FIG. 2A and FIG. 2B, FIG. 1 is a schematic diagram of a structure of a human eye, FIG. 2A is a schematic diagram of a physiological characteristic measuring device according to an embodiment of the invention, and FIG. 2B is a schematic diagram of a physiological characteristic measuring method according to an embodiment of the invention. As shown in FIG. 1, there are an iris 12, an aqueous humor 13, and a cornea 14 in front of a lens 11 of a human eye 10. The physiological characteristic measuring device 1 of the embodiment may be used to measure a glucose concentration in the aqueous humor 13. Specifically, natural glucose in human body and in nature is only in the form of a right-handed structure. Therefore, when polarized light passes through a glucose solution, a direction of electric field thereof shall be rotated. Therefore, the physiological characteristic measuring device 1 according to the embodiment of the invention uses polarized light to measure the glucose concentration in the aqueous humor 13 in response to the above phenomenon.

Referring to FIG. 2A, the physiological characteristic measuring device 1 includes a light source module 200, a sensing module 100, and a processing unit 300.

The light source module 200 includes a light source 201 and a linear polarizer 202. The light source 201 is configured to provide light L0. After passing through the linear polarizer 202, the light L0 forms incident linear polarized light L1. The incident linear polarized light L1 has a first linear polarization P1. A wavelength of the incident linear polarized light L1 falls within a range of 460 nm to 940 nm to prevent a volume of water in the aqueous humor 13 from changing or absorbing the incident linear polarized light L1 of a specific wavelength, thereby changing a signal intensity, but the invention is not limited thereto. After the incident linear polarized light L1 enters the eye 10, it is reflected on a surface of the lens 11 to generate a light to be measured L2.

The sensing module 100 is configured to sense the light to be measured L2 from the eye 10. The light source 201 may include a laser source and a light-emitting diode, but the invention is not limited thereto. The sensing module 100 includes a linear polarizer 103, a linear polarizer 104, a beam splitter 105, a sensing device 101 and a sensing device 102, where the beam splitter 105 is disposed on a path of the light to be measured L2, and the sensing devices 101 and 102 are connected to the processing unit 300.

In some embodiments, the processing unit 300 is, for example, a central processing unit (CPU), a microprocessor (microprocessor), a digital signal processor (DSP), a programmable controller, a programmable logic device (PLD), or other similar devices or a combination of these devices, which is not limited by the invention. In addition, in some embodiments, each function of the processing unit 300 may be implemented as a plurality of program codes. These program codes may be stored in a memory and executed by the processing unit 300. Alternatively, in an embodiment, each function of the processing unit 300 may be implemented as one or more circuits. The invention does not limit the implementation of each function of the processing unit 300 by software or hardware.

The incident linear polarized light L1 and the light to be measured L2 define an optical plane (i.e., an X-Y plane), and the first linear polarization P1 is parallel to the optical plane. A part of the light to be measured L2 is formed into a first light to be measured L21 after passing through the beam splitter 105. The beam splitter 105 reflects a part of the light to be measured L2 to generate a second light to be measured L22. The first light to be measured L21 passes through the polarizer 103 to generate a linear polarized light L23, and the linear polarized light L23 has a second linear polarization P2. The second light to be measured L22 passes through the polarizer 104 to generate a linear polarized light L24, and the linear polarized light L24 has a third linear polarization P3. A linear polarization direction of the linear polarized light L23 is parallel to the optical plane (parallel to the X-Y plane), and a linear polarization direction of the linear polarization light L24 is perpendicular to the optical plane (perpendicular to the X-Y plane), as shown in FIG. 2A. In general, the first linear polarization P1 and the second linear polarization P2 are all parallel to the X-Y plane, and the third linear polarization P3 is perpendicular to the X-Y plane. In other words, the first linear polarization P1 and the second linear polarization P2 are both horizontal polarizations with respect to the X-Y plane, and the third linear polarization P3 is vertical polarization with respect to the X-Y plane. It should be noted that the first linear polarization P1 is not limited to being parallel to the X-Y plane. In some embodiments, the first linear polarization P1 is perpendicular to the X-Y plane.

Furthermore, the sensing device 101 and the sensing device 102 are respectively configured to measure a light intensity I1 of the linear polarized light L23 and a light intensity I2 of the linear polarized light L24. The processing unit 300 may calculate a ratio of the light intensity I2 to the light intensity I1, which is a ratio of an electric field intensity of the light to be measured L2 in a vertical direction with respect to the X-Y plane and an electric field intensity in a horizontal direction with respect to the X-Y plane. The processing unit 300 further calculates an arc-tangent function of the intensity ratio, which is an angle θ (equation 1) of an electric field of the light to be measured L2 relative to the X-Y plane, i.e., a rotation angle θ of the electric field of the horizontally polarized light L1 relative to the X-Y plane after passing through the eye. The above rotation angle θ may change according to the glucose concentration in the aqueous humor 13. In other words, the glucose concentration in the aqueous humor 13 may be measured according to the above rotation angle θ.

$$θ = {tan}^{- 1} I 2 / I 1 \qquad Equation 1$$

It should be noted that, in some embodiments, the first linear polarization P1 is perpendicular to the X-Y plane. In this case, the light intensity I1 in the equation 1 is the light intensity of the linear polarized light L24, and the light intensity I2 is the light intensity of the linear polarized light L23. The arc-tangent function shown in equation 1 is the rotation angle θ of the electric field of the vertical polarized light L1 with respect to the X-Y plane after passing through the eye.

The physiological characteristic measuring device 1 may further include a camera device 400, which is connected to the processing unit 300. The camera device 400 may capture an image of the eye 10.

In some embodiments, the camera device 400 may be used to locate the physiological characteristic measuring device 1. In such a situation, an optical axis C1 of the camera device 400 is configured on the optical plane (X-Y plane) to improve positioning accuracy, where there is an angle between the incident linear polarized light L1 and the optical axis C1 of the camera device 400, and the angle may fall within a range of 0 degree to 90 degrees. Specifically, the physiological characteristic measuring device 1 may be implemented as a head-mounted device, and further includes a moving mechanism (not shown). The moving mechanism is connected to the processing unit 300, and the processing unit 300 determines whether the physiological characteristic measuring device 1 is worn in a proper position according to the image provided by the camera device 400. If the characteristic measuring device 1 is not worn in a proper position, the processing unit 300 may control the moving mechanism to move the light source module 200, the sensing module 100 and the camera device 400 to improve the accuracy of the measurement.

Referring to FIG. 2B, when an incident angle θ₁ and a position of the incident linear polarized light L1 generated by the light source 201 on a surface of the cornea 14 meet a following conditional expression (equation 2), the incident linear polarized light L1 may reach a vertex 110 of the lens 11 and may be further formed into the light to be measured L2 that enters the sensing device 101 and the sensing device 102.

$${θ}_{3} = {sin}^{- 1} ( \frac{sin {θ}_{1}}{1.33} ) \qquad Equation 2$$

$$α = {cos}^{- 1} [ \frac{{L}_{1}^{2} + {L}_{2}^{2} - {( {L}_{1} cos {θ}_{3} - \sqrt{{( {L}_{1} cos {θ}_{3} )}^{2} - ( {L}_{1}^{2} - {L}_{2}^{2} )} )}^{2}}{2 {L}_{1} {L}_{2}} ]$$

In equation 2 and FIG. 2B, θ₃ is a refractive angle of the incident linear polarized light L1 when it enters the aqueous humor 13, 1.33 is a refractive index of the aqueous humor 13, L₃ is a distance of the incident linear polarized light L1 between an inner layer of the cornea 14 and the vertex 110 of the lens 11, L₁ is a radius of curvature of the inner layer of the cornea 14, L₂ is a distance from the vertex 110 of the lens 11 to a center O of the radius of curvature of the inner layer of the cornea 14, and α is an included angle between a line of the incident linear polarized light L1 from the inner layer of the cornea 14 to the center O of the radius of curvature and a line from the vertex 110 of the lens 11 to the center O of the radius of curvature.

In order to fully illustrate various implementations of the invention, other embodiments of the invention will be described below. It must be noted that the following embodiments use the component referential numbers and some contents of the previous embodiments, where the same referential numbers are used to represent the same or similar components, and the description of the same technical contents is omitted. For the description of the omitted parts, please refer to the previous embodiments, which will not be repeated in the following embodiments.

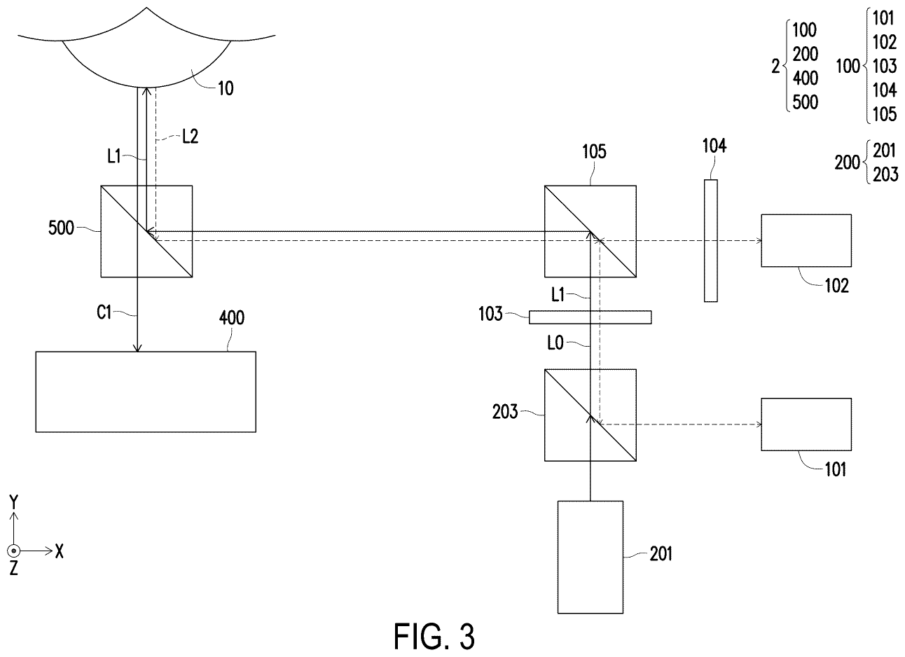

FIG. 1 and FIG. 3, FIG. 3 is a schematic diagram of a physiological characteristic measuring device according to an embodiment of the invention.

A physiological characteristic measuring device 2 includes a light source module 200, a sensing module 100, a beam splitter 500, and a processing unit (not shown).

The light source module 200 includes a light source 201 and a beam splitter 203, and is configured to provide light L0. The sensing module 100 includes a linear polarizer 103, a linear polarizer 104, a beam splitter 105, a sensing device 101 and a sensing device 102. The sensing device 101 and the sensing device 102 are connected to the processing unit. An absorption axis of the linear polarizer 103 is perpendicular to an absorption axis of the linear polarizer 104.

The light L0 is formed into incident linear polarized light L1 after passing through the linear polarizer 103, and a polarization direction of the incident linear polarized light L1 is parallel to the X-Y plane. After the incident linear polarized light L1 enters the eye 10, it is reflected on the surface of the lens 11 to generate the light to be measured L2, where the incident linear polarized light L1 and the light to be measured L2 overlap. For the convenience of understanding, in FIG. 3, the incident linear polarized light L1 and the light to be measured L2 are illustrated as not overlapping.

After being reflected by the beam splitter 500, the light to be measured L2 partially penetrates through and is partially reflected by the beam splitter 105. The light to be measured L2 that penetrates through the linear polarizer 104 enters the sensing device 102, and the sensing device 102 measures the light intensity I2 of the light to be measured L2. The light to be measured L2 that penetrates through the linear polarizer 103 is reflected by the beam splitter 203 and enters the sensing device 101, and the sensing device 101 measures the light intensity I1 of the light to be measured L2. The processing unit calculates the ratio of the light intensity I2 and the light intensity I1 according to the above equation 1, which is the ratio of the electric field intensity of the light to be measured L2 from the eye in the vertical direction relative to the X-Y plane and the electric field intensity of the same in the horizontal direction relative to the X-Y plane. The processing unit further calculates an arc-tangent function of the intensity ratio, which is an angle θ (equation 1) of the electric field of the light to be measured L2 from the eye relative to the X-Y plane, i.e., the rotation angle θ of the electric field of the horizontally polarized light L1 with respect to the X-Y plane after passing through the eye. The glucose concentration in the aqueous humor 13 may be measured according to the above rotation angle θ.

In the embodiment, both of the incident linear polarized light L1 and the light to be measured L2 pass through the beam splitter 105 and the beam splitter 500. By configuring the beam splitter 105 and the beam splitter 500, a design margin of the physiological characteristic measuring device 2 is improved.

In addition, the physiological characteristic measuring device 2 may further include a camera device 400, and the incident linear polarized light L1 and the light to be measured L2 both overlap the optical axis C1 of the camera device 400. The function of the camera device 400 is as described in the above embodiment, and detail thereof is not repeated here.

Referring to FIG. 1 and FIG. 2A again, in another embodiment of the invention, the electric field rotation angle θ may be measured by using the physiological characteristic measuring device 1 in combination with another measuring method, where an absorption axis of the linear polarizer 202 is perpendicular to the absorption axis of the linear polarizer 104 and perpendicular to the X-Y plane, as shown in FIG. 2A. Specifically, in the embodiment, the linear polarizer 202 of the physiological characteristic measuring device 1 is not on the path of the light L0 in a first time period (for example, is moved out of the path of the light L0), and is arranged on the path of the light L0 in a second time period (as shown in FIG. 2A). The sensing device 102 measures a light intensity I3 in the first time period and measures the light intensity I4 in the second time period. Since the electric field rotation angle θ caused by the glucose in the aqueous humor 13 is very small, the rotation angle θ may be defined by equation 3 by using an approximate method:

$$θ = {sin}^{- 1} I 4 / I 3$$

In another embodiment of the invention, the absorption axis of the linear polarizer 202 is perpendicular to the absorption axis of the linear polarizer 103 and parallel to the X-Y plane. The linear polarizer 202 of the physiological characteristic measuring device 1 is not on the path of the light L0 in the first time period, and is arranged on the path of the light L0 in the second time period. The sensing device 101 measures a light intensity I5 in the first time period and measures a light intensity I6 in the second time period. Since the electric field rotation angle θ caused by the glucose in the aqueous humor 13 is very small, the rotation angle θ may be defined by equation 4 by using an approximate method:

$$θ = {sin}^{- 1} I 6 / I 5$$

Based on the above, the physiological characteristic measuring device provided by the embodiment of the invention utilizes optical rotation of glucose and uses polarized light to measure a glucose concentration in the aqueous humor, and the blood glucose concentration of the testee may be obtained based on the relationship between the glucose concentration in the aqueous humor and the blood glucose concentration. Comparing the results with other invasive measuring devices, the physiological characteristic measuring device provided by the embodiment of the invention does not require drawing blood and is easy to carry around.

What is claimed is:

1. A physiological characteristic measuring device, adapted to be arranged in front of an eye, comprising:
a light source module, configured to provide incident linear polarized light toward the eye, wherein the incident linear polarized light has a first linear polarization;
a sensing module, wherein when the incident linear polarized light enters the eye, the sensing module is configured to sense light to be measured from the eye; and
a processing unit, connected to the sensing module, wherein the processing unit is configured to provide a light intensity ratio, the light intensity ratio is a ratio of a light intensity of a first linear polarized light of the light to be measured to a light intensity of a second linear polarized light of the light to be measured, the first linear polarized light has a second linear polarization, the second linear polarized light has the first linear polarization, and the second linear polarization is perpendicular to the first linear polarization,
wherein the processing unit provides a physiological characteristic measuring value according to an arc-tangent function of the light intensity ratio.

2. The physiological characteristic measuring device as claimed in claim 1, wherein the incident linear polarized light and the light to be measured define an optical plane, the first linear polarization is parallel to the optical plane, and the second linear polarization is perpendicular to the optical plane.

3. The physiological characteristic measuring device as claimed in claim 1, wherein the incident linear polarized light and the light to be measured define an optical plane, the first linear polarization is perpendicular to the optical plane, and the second linear polarization is parallel to the optical plane.

4. The physiological characteristic measuring device as claimed in claim 1, wherein the light source module comprises a laser source and a first linear polarizer, and the laser source provides laser light to be incident to the first linear polarizer.

5. The physiological characteristic measuring device as claimed in claim 1, wherein the sensing module comprises a beam splitter, a first sensing device and a second sensing device, the beam splitter is disposed on a path of the light to be measured, and the first sensing device and the second sensing device are connected to the processing unit.

6. The physiological characteristic measuring device as claimed in claim 1, further comprising a camera device connected to the processing unit, wherein the incident linear polarized light and the light to be measured define an optical plane, the camera device is configured to photograph the eye, and an optical axis of the camera device is located on the optical plane.

7. The physiological characteristic measuring device as claimed in claim 6, further comprising a moving mechanism connected to the processing unit, wherein the processing unit controls the moving mechanism to move the light source module, the sensing module and the camera device according to an image provided by the camera device.

8. The physiological characteristic measuring device as claimed in claim 6, wherein the light source module comprises a laser source and a first linear polarizer, the laser source provides laser light, the laser light is incident on the first linear polarizer to generate the incident linear polarized light, there is an angle between the incident linear polarized light and the optical axis of the camera device, and the angle falls within a range of 0 to 90 degrees.

9. The physiological characteristic measuring device as claimed in claim 1, wherein a wavelength of the incident linear polarized light falls within a range of 460 nm to 940 nm.

10. The physiological characteristic measuring device as claimed in claim 1, wherein the incident linear polarized light and the light to be measured overlap with each other.

11. The physiological characteristic measuring device as claimed in claim 1, further comprising at least one beam splitter, wherein both of the incident linear polarized light and the light to be measured pass through the at least one beam splitter.

12. The physiological characteristic measuring device as claimed in claim 11, wherein a number of the at least one beam splitter is 2.

13. The physiological characteristic measuring device as claimed in claim 6, wherein both of the incident linear polarized light and the light to be measured overlap the optical axis of the camera device.

14. A physiological characteristic measuring device, adapted to be arranged in front of an eye, comprising:
a light source module, comprising a light source and a first linear polarizer, wherein the light source is configured to provide incident light toward the eye, the incident light comprises first incident light in a first time period and second incident light in a second time period, in the first time period, the first linear polarizer is not on a path of the incident light, and in the second time period, the first linear polarizer is on the path of the incident light;
a sensing module, comprising a second linear polarizer, wherein when the incident light enters the eye, the sensing module is configured to sense light to be measured from the eye to generate a light intensity of the light to be measured, the second linear polarizer is located on a path of the light to be measured, and an absorption axis of the first linear polarizer is perpendicular to an absorption axis of the second linear polarizer; and
a processing unit, connected to the sensing module, wherein the processing unit is configured to provide a light intensity ratio, the light intensity ratio is a ratio of the light intensity of the light to be measured in the second time period to the light intensity of the light to be measured in the first time period, wherein
the processing unit provides a physiological characteristic measuring value according to an arc-sine function of the light intensity ratio.

15. The physiological characteristic measuring device as claimed in claim 14, wherein the incident light and the light to be measured define an optical plane, the absorption axis of the first linear polarizer is parallel to the optical plane, and the absorption axis of the second linear polarizer is perpendicular to the optical plane.

16. The physiological characteristic measuring device as claimed in claim 14, wherein the incident light and the light to be measured define an optical plane, the absorption axis of the first linear polarizer is perpendicular to the optical plane, and the absorption axis of the second linear polarizer is parallel to the optical plane.
